(19) United States
(12) Patent Application Publication
(10) Pub. No.: US 2013/0338545 A1
(43) Pub. Date: Dec. 19, 2013

(54) ULTRASOUND SKIN TREATMENT

(71) Applicants: Haim Azhari, Doar-Na Misgav (IL); Liat Tsoref, Tel Aviv (IL); Gregory Krasnov, Haifa (IL); Yossi Gross, Moshav Mazor (IL)

(72) Inventors: Haim Azhari, Doar-Na Misgav (IL); Liat Tsoref, Tel Aviv (IL); Gregory Krasnov, Haifa (IL); Yossi Gross, Moshav Mazor (IL)

(73) Assignee: SLENDER MEDICAL LTD., Herzliya (IL)

(21) Appl. No.: 13/994,246

(22) PCT Filed: Dec. 14, 2011
(86) PCT No.: PCT/IL11/00942

§ 371 Date: Sep. 5, 2013

Related U.S. Application Data
(60) Provisional application No. 61/422,887, filed on Dec. 14, 2010.

(51) Int. Cl. A61N 7/00

(52) U.S. Cl. CPC A61N 7/00
USPC 601/2

(57) ABSTRACT

Apparatus (20) is provided comprising a skin-application portion (40), configured to move across skin of a subject. At least one acoustic element (50) is coupled to the skin-application portion (40) and configured to be placed in acoustic contact with the skin, and configured to apply ultrasound energy to the skin. The apparatus (20) additionally comprises circuitry configured to generate a current responsive to motion of the skin-application portion (40); and a control unit (10), which is configured to receive the current, to determine, responsive thereto, whether the skin-application portion (40) is moving with respect to the skin, and to drive the acoustic element (50) to apply the ultrasound energy to the skin responsive to determining that the skin-application portion (40) is moving with respect to the skin. Other embodiments are also described.

### CROSS-REFERENCES TO RELATED APPLICATIONS

The present application claims the priority of U.S. Provisional Application 61/422,887 to Azhari et al., entitled, “Ultrasound skin treatment,” filed Dec. 14, 2010, which is incorporated herein by reference.

### FIELD OF THE APPLICATION

The present invention relates generally to skin treatment and particularly to methods and apparatus for skin rejuvenation by application of ultrasound energy.

### BACKGROUND OF THE APPLICATION

Skin, the body's largest organ, is composed of multiple layers. The outer layer, epidermis, is divided into several sublayers. Beneath the epidermis lies the dermis skin layer, which is composed of two layers, the upper papillary layer and the lower reticular layer.

A major structural component of the dermis skin layer is collagen, a fibrous protein, which contributes to skin strength and elasticity. As such, collagen formation, and in contrast age-related collagen decline, leads to changes in mechanical properties of the skin, such as texture and resilience. Thermal treatment of the skin can lead to thermal shrinkage of collagen, which occurs by the dissociation of heat-sensitive bonds of the collagen molecule. Thermal denaturing of collagen typically results in a tightening effect of the skin.

Visible effects of aging or damage of the skin are disturbing to many individuals and therefore methods for rejuvenation of maturing or damaged skin are of interest. Some skin rejuvenation methods include application of energy to heat selected areas of the skin in order to obtain an improvement in the appearance of the treated skin.

### SUMMARY OF APPLICATIONS

Some applications of the present invention comprise apparatus and methods for skin rejuvenation by applying treatment energy to the skin and monitoring application of the energy thereto. As provided by some applications of the present invention, apparatus comprising a skin-application portion is moved across skin of a subject. Typically, at least one acoustic element, e.g., an ultrasound transducer, is coupled to the skin-application portion and is placed in acoustic contact with the skin of the subject. The acoustic element applies ultrasound energy, e.g., high intensity focused ultrasound (HIFU) energy, to the skin. The skin-application portion further comprises circuitry which generates a current responsive to motion of the skin-application portion. A control unit receives the current and determines, in response thereto, whether the skin-application portion is moving with respect to the skin, and drives the acoustic element to apply the ultrasound energy to the skin responsive to determining that the skin-application portion is moving with respect to the skin.

For some applications, the apparatus comprises a pressure sensor which generates a pressure sensor signal responsive to contact of the skin-application portion with the skin. The control unit receives the pressure sensor signal and determines, responsive thereto, a degree of contact of the skin-application portion with the skin, and controls application of ultrasound energy from the acoustic element to the skin responsive to determining the contact of the skin-application portion with the skin.

There is therefore provided in accordance with some application of the present invention, apparatus, including:
a skin-application portion, configured to move across skin of a subject;
at least one acoustic element coupled to the skin-application portion and configured to be placed in acoustic contact with the skin, and configured to apply ultrasound energy to the skin;
circuitry configured to generate a current responsive to motion of the skin-application portion; and
a control unit, which is configured to receive the current, to determine, responsive thereto, whether the skin-application portion is moving with respect to the skin, and to drive the acoustic element to apply the ultrasound energy to the skin responsive to determining that the skin-application portion is moving with respect to the skin.

For some applications, the apparatus includes a pressure sensor configured to generate a pressure sensor signal responsive to contact of the skin-application portion with the skin, and the control unit is adapted to receive the pressure sensor signal, to determine, responsive thereto, a degree of contact of the skin-application portion with the skin, and to control application of ultrasound energy from the acoustic element to the skin responsive to determining the contact of the skin-application portion with the skin.

For some applications, at least one rotating element is coupled to the circuitry.

For some applications, the rotating element includes a wheel that is rotatable due to the skin-application portion being moved with respect to the skin, while in contact with the skin.

For some applications, the wheel is permitted to turn in a first direction, and inhibited from turning in a second direction, opposite to the first direction.

For some applications, the control unit is configured to allow the application of the ultrasound energy when the wheel turns in a first direction, and to inhibit the application of the ultrasound energy when the wheel is turning in a second direction, opposite to the first direction.

For some applications, the circuitry includes a sensor selected from the group consisting of: an optical sensor, a magnetic sensor, and an acceleration sensor.

For some applications, the skin-application portion is configured to move across the skin in a continuous manner.

For some applications, the acoustic element includes an ultrasound transducer configured to apply the ultrasound energy as high intensity focused ultrasound (HIFU) energy.

For some applications, the apparatus is configured to monitor a temperature of the skin and to regulate the application of the ultrasound energy in response thereto.

For some applications, the acoustic element includes a piezoelectric element.

For some applications, a conductive material is coupled to a plurality of distinct areas of the same piezoelectric element.

For some applications, a first one of the plurality of distinct areas is configured to receive ultrasound energy transmitted by a second one of the distinct areas, and wherein the control unit is configured to determine an indication of a temperature of the skin, in response to a parameter of the received energy, and to regulate the application of the ultrasound energy in response to the parameter.

For some applications, the parameter is time of flight, and the control unit is configured to monitor the time of flight and to regulate the application of the ultrasound energy in response thereto.

For some applications, the parameter is selected from the group consisting of: an amplitude of the ultrasound energy applied by the ultrasound transducer, and a sub-harmonic of the received ultrasound energy, and the control unit is configured to monitor the selected ultrasound parameter and to regulate the application of the ultrasound energy in response to the selected parameter.

For some applications, the at least one acoustic element includes at least two acoustic elements, respectively including an ultrasound transmitter and an ultrasound receiver.

For some applications, the ultrasound receiver is configured to receive reflected ultrasound energy and the apparatus is configured to monitor an ultrasound parameter of the reflected energy.

There is further provided in accordance with some applications of the present invention a method including:
moving an acoustic element across skin of a subject, while the acoustic element is in acoustic contact with the skin;
activating the acoustic element to apply ultrasound energy to the skin;
generating a current responsive to motion of the acoustic element across the skin;
determining, responsive to the current, whether the acoustic element is moving with respect to the skin; and
controlling application of ultrasound energy from the acoustic element to the skin responsive to determining that the acoustic element is moving with respect to the skin.

For some applications, the method includes operating a pressure sensor to generate a pressure sensor signal responsive to the contact of the acoustic element with the skin, and determining further includes determining, in response to the pressure signal, a degree of contact of the acoustic element with the skin, and controlling application of ultrasound energy from the acoustic element to the skin responsive to determining the contact of the acoustic element with the skin.

For some applications, moving the acoustic element includes moving the acoustic element in a continuous manner over the skin.

For some applications, activating includes activating the ultrasound transducer to apply the ultrasound energy as high intensity focused ultrasound (HIFU) energy.

For some applications, the method includes monitoring a temperature of the skin in response to activating the acoustic element to apply ultrasound energy to the skin.

For some applications, the method includes applying a substance to the skin, the substance including an agent; and
facilitating delivery of the agent into the skin by the application of the ultrasound energy.

For some applications, the method includes applying a substance to the skin, the substance including an agent; and
activating the agent by the application of the ultrasound energy.

For some applications, the substance includes microcapsules including the agent, and activating the agent by the application of the ultrasound energy includes releasing the agent from the microcapsules due to rupture of the microcapsules by the ultrasound energy.

For some applications, activating the agent by the application of the ultrasound energy includes elevating a temperature of the skin to a temperature that is above 37 C.

For some applications, the substance includes metal nano-particles, and activating the agent by the application of the ultrasound energy includes raising a temperature of the substance by reflecting the ultrasound energy from the metal particles.

For some applications, the method includes injecting a substance through the skin, the substance including a heat-sensitive agent; and
alerting a mechanical property of the agent by the application of the ultrasound energy.

There is additionally provided in accordance with some applications of the present invention apparatus, including:
a skin-application portion, configured to move across skin of a subject;
at least one energy source coupled to the skin-application portion and configured to be placed in contact with the skin, and configured to apply energy to the skin;
circuitry configured to generate a current responsive to motion of the skin-application portion; and
a control unit, which is configured to receive the current, to determine, responsive thereto, whether the skin-application portion is moving with respect to the skin, and to drive the energy source to apply the energy to the skin responsive to determining that the skin-application portion is moving with respect to the skin.

For some applications, the energy source includes an energy source selected from a group consisting of; a microwave energy source, a radio frequency energy source, an optical energy source and an electrical energy source.

There is still additionally provided in accordance with some applications of t le present invention a substance, including:
an agent; and
microcapsules containing the agent and configured to release the agent into the skin by rupturing in response to ultrasound energy.

For some applications, the agent includes a therapeutic agent.

For some applications, the agent includes a cosmetic agent.

For some applications, the substance includes a carrier in which the microcapsules are disposed, the carrier selected from the group consisting of: a gel and a cream.

There is yet additionally provided in accordance with some applications of the present invention a substance for application to skin of a subject, including:
metal particles configured to raise a temperature of the substance by reflecting ultrasound energy applied to the skin; and
an agent configured to assume an active state thereof in response to the raise in the temperature of the substance.

For some applications, the substance includes a carrier in which the metal particles and agent are disposed, the carrier selected from the group consisting of: a gel and a cream.

For some applications, the agent includes a therapeutic agent.

For some applications, the agent includes a cosmetic agent.

The present invention will be more fully understood from the following detailed description of applications thereof, taken together with the drawings, in which:

### BRIEF DESCRIPTION OF THE DRAWINGS

FIGS. 1A-B are schematic illustrations of apparatus for skin treatment, in accordance with some applications of the present invention;

FIG. 2 is a schematic illustration of an acoustic element positioned in contact with the skin surface, in accordance with some applications of the present invention;

FIG. 3 is a schematic illustration of a configuration of the apparatus for skin treatment, in accordance with some applications of the present invention;

FIGS. 4A-B are schematic illustrations of configurations of the apparatus for skin treatment of FIGS. 1-2, positioned in contact with the skin surface, in accordance with some applications of the present invention;

FIGS. 5A-C are schematic illustrations of configurations of piezoelectric elements for use with the apparatus for skin treatment, in accordance with some applications of the present invention;

FIG. 6 is a schematic illustration of an additional configuration of the apparatus for skin treatment, in accordance with some applications of the present invention; and

FIG. 7 is a schematic illustration of yet another configuration of the apparatus for skin treatment, in accordance with some applications of the present invention.

### DETAILED DESCRIPTION OF APPLICATIONS

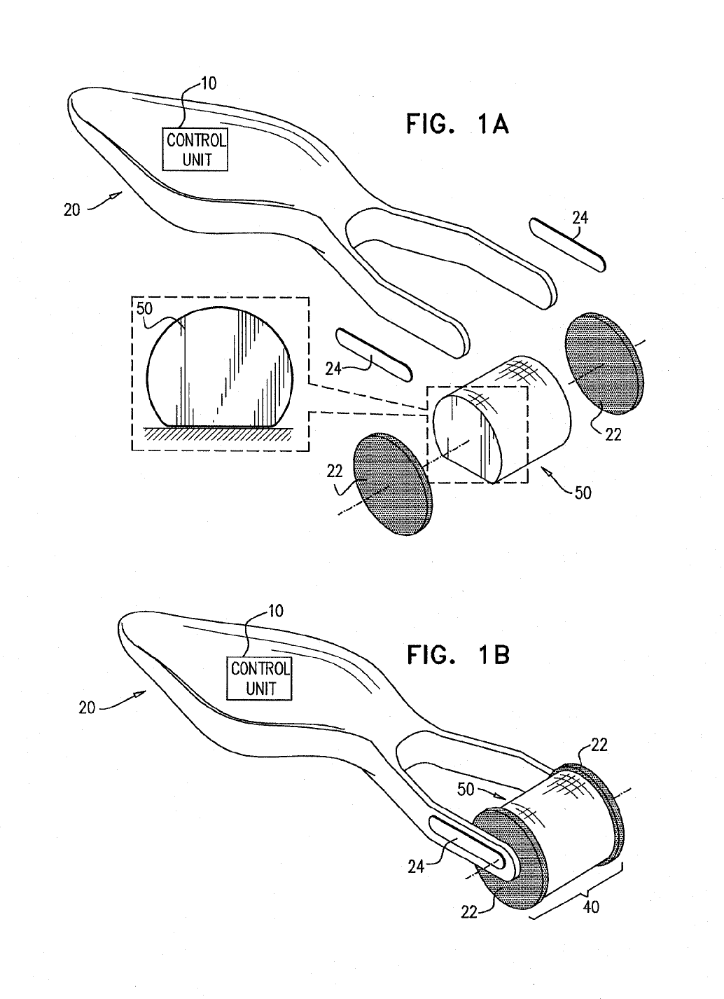

FIGS. 1A-B are schematic illustrations of apparatus 20 for skin treatment, in accordance with some applications of the present invention. Apparatus 20 typically comprises handheld apparatus comprising a skin-application portion 40 that is moved across a selected region of skin of a subject, e.g., skin on a face of the subject. Typically skin-application portion 40 is moved across the skin in a continuous manner. Skin-application portion 40 is typically but not necessarily disposable and/or sterile. Typically, skin application portion 40 comprises at least one acoustic element, e.g., an ultrasound transducer 50, that is placed in acoustic contact with the skin of the subject. Transducer 50 applies ultrasound energy, e.g., high intensity focused ultrasound (HIFU) energy, to the skin. Application of ultrasound energy to the skin surface with subsequent heating of underlying skin layers typically leads to collagen regeneration and remodeling, resulting in skin tightening and rejuvenation.

For some applications, a substance (in a carrier such as a gel or a cream) which may comprise a therapeutic and/or cosmetic agent, is applied to skin-application portion 40 and/or to a portion of a skin surface designated for treatment. Typically, the substance also serves as an acoustic coupling medium between ultrasound transducer 50 and the skin surface. Typically, the ultrasound energy emitted from ultrasound transducer 50 enhances delivery to the skin of the therapeutic and/or cosmetic agents in the substance. Additionally or alternatively, the ultrasound energy mechanically or thermally activates the therapeutic and/or cosmetic agents in the substance. For example, the substance may comprise microcapsules which contain the therapeutic and/or cosmetic agents, and the ultrasound energy transmitted from transducer 50 releases the therapeutic and/or cosmetic agents from the microcapsules due to rupture of the microcapsules. For some applications, the therapeutic and/or cosmetic agents in the substance which are applied to the skin surface of the subject are activated at a temperature that is higher than 37 C. For such applications, the ultrasound energy transmitted from transducer 50 elevates the temperature of the tissue (e.g., to 42 C) thereby activating the therapeutic and/or cosmetic agents in the substance. For some applications, the substance comprises metal (e.g. gold) nano-particles. The ultrasound energy that is reflected from the metal nano-particles typically raises the temperature of the substance, thus activating it.

Apparatus 20 is typically configured to monitor application of the energy to the skin, e.g., such that the applied energy generally does not cause undesired effects of overheating of the skin and underlying layers. Alternatively or additionally, skin-application portion 40 typically comprises circuitry which generates a current responsive to motion of the skin-application portion 40. For some applications, the circuitry is coupled to at least one rotating element 22, e.g., a wheel, which is rotatable due to the skin-application portion being moved with respect to the skin, while in contact with the skin. The circuitry generates a current responsive to motion of the skin-application portion across the skin.

A control unit 10 receives the current from the circuitry and determines, in response thereto, whether skin-application portion 40 is moving with respect to the skin, and drives the acoustic element to apply the ultrasound energy to the skin while skin-application portion 40 is moving with respect to the skin. Control unit 10 is configured to alter application of the energy from transducer 50, when motion of skin-application portion 40 across the skin is altered. For example, application of energy from transducer 50 may be discontinued or reduced when skin-application portion 40 is moving slowly or is not moving across the skin of the subject, in order to prevent excessive heating of the skin.

Additionally or alternatively, apparatus 20 is configured to allow motion of skin-application portion 40 substantially in a first direction, in order to generally avoid multiple applications of ultrasound energy to a particular skin area which may result in excessive heating of that area. For some applications, rotating element 22 is permitted to turn in the first direction, and is inhibited from turning in a second direction, opposite to the first direction, thereby facilitating motion of skin-application portion 40 in the first direction only. Additionally, control unit 10 allows the application of the ultrasound energy when rotating element 22 turns in the first direction, and inhibits the application of the ultrasound energy when rotating element 22 is turned in the second direction.

For some applications, apparatus 20 comprises a pressure sensor 24, which generates a pressure sensor signal responsive to contact of skin-application portion 40 with the skin. Control unit 10 receives the pressure sensor signal and determines, responsive thereto, a degree of physical contact of skin-application portion 40 with the skin, and controls application of ultrasound energy from transducer 50 to the skin responsive to determining the contact of skin-application portion 40 with the skin. Accordingly, control unit 10 is configured to alter application of the energy from transducer 50, when contact of skin-application portion 40 with the skin is altered. For example, application of energy from transducer 50 may be discontinued when skin-application portion 40 is sensed by pressure senor 24 as not being in sufficient contact with the skin of the subject.

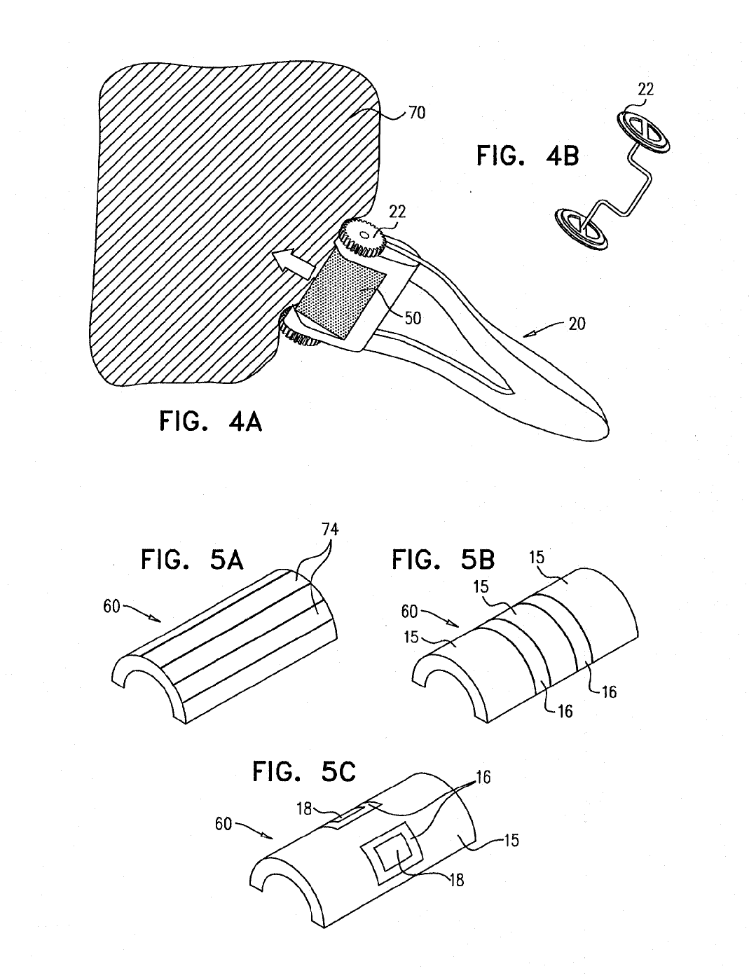

Alternatively, rotating element 22 comprises pressure sensing functionality that operates like pressure sensor 24 (as described hereinbelow with reference to FIG. 4B.)

Additionally to sensing physical contact of skin-application portion 40 with the skin as an indication of sufficient acoustic contact, apparatus 20 is typically configured to verify sufficient acoustic contact of skin-application portion 40 with the skin, by receiving an echo of transmitted ultrasound waves. For some applications, transducer 50 transmits low intensity ultrasound energy and receives its echo. The amplitude of the echo is typically below a desired threshold if the acoustic contact is not sufficient. When the acoustic contact is not sufficient, control unit 10 inhibits transducer SO from transmitting treatment energy, e.g., high intensity focused ultrasound (HIFU) energy, to the skin. It is to be noted that, any form of monitoring described herein, e.g., motion, physical contact and acoustic contact monitoring, or a combination thereof, may be used in accordance with applications of the present invention.

It is to be noted that apparatus 20 for skin treatment may comprise an alternative energy source which applies energy to the skin, e.g., a microwave energy source, and/or a radio frequency energy source, and/or optical energy source and/or an electrical energy source or a combination thereof. Additionally or alternatively, any of these energy sources may be used in combination with ultrasound transducer 50.

Reference is made to FIG. 2, which is a schematic illustration of transducer 50 positioned in contact with skin surface 70, in accordance with some applications of the present invention. Apparatus 20 is typically configured to focus energy transmission to a particular area of the skin in order to enable treatment of a small focal zone 72. Transducer 50 comprises a piezoelectric element 60. For some applications, piezoelectric element 60 is shaped to define an arch, and the ultrasound energy emitted from element 60 is focused such that focal zone 72 is disposed beneath the outer layer of the skin, the epidermis, so as to avoid damage to the epidermis. Piezoelectric element 60 may be surrounded by a cooling fluid that is present within transducer 50 and typically removes excess heat from the transducer and from the outer layer of the skin.

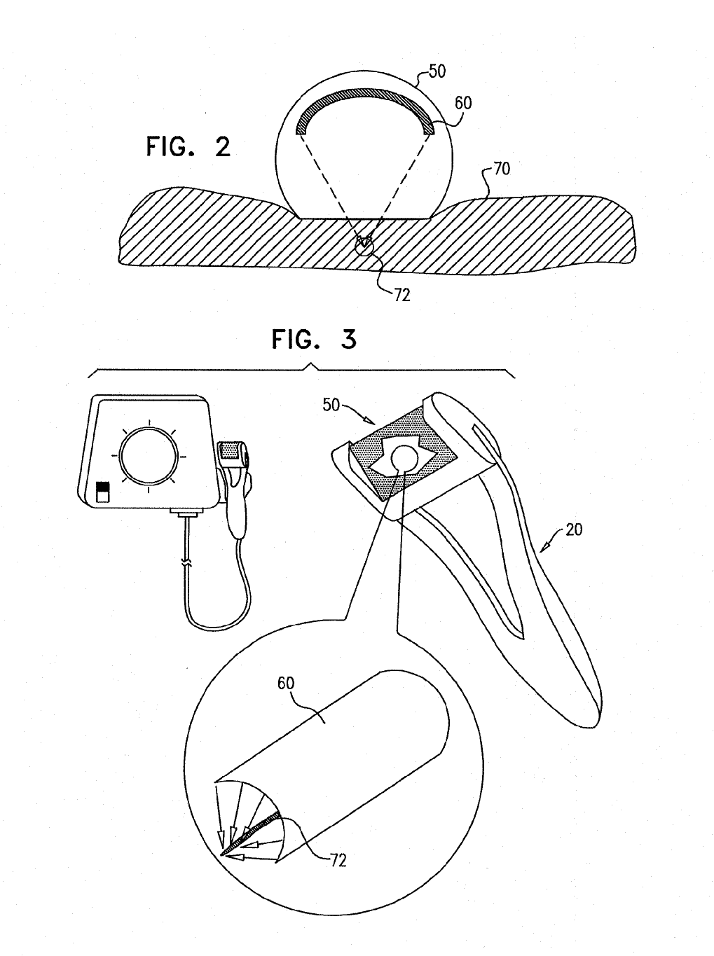

Reference is made to FIG. 3, which is a schematic illustration of apparatus 20 for skin treatment, as provided by some applications of the present invention. As described hereinabove with reference to FIG. 1, apparatus 20 comprises motion sensing functionality, e.g., a rotating element 22, in order to facilitate proper application of ultrasound energy to treat skin of a subject. Additionally or alternatively, apparatus 20 comprises other sensing modalities, e.g., an optical sensor (like that used in a computer mouse) and/or a magnetic sensor and/or an acceleration sensor or a combination thereof.

FIG. 3 shows an exploded view of arched piezoelectric element 60 transmitting ultrasound energy toward focal zone 72.

Reference is made to FIG. 4A, which is a schematic illustration of apparatus 20 positioned in contact with skin surface 70, in accordance with some applications of the present invention. For some applications rotating element 22 is shaped to define a cog-wheel.

Typically, skin-application portion 40 comprises transducer 50, which is configured to transmit ultrasound energy to the skin. For some applications, transducer 50 comprises a membrane, e.g., a polyurethane membrane, represented in FIG. 4A by the dotted area, covering a piezoelectric element. The membrane typically facilitates transmission of ultrasound energy with a reduced degree of energy loss.

FIG. 4B shows a configuration of a pair of non-cog rotating elements 22, which is coupled to circuitry configured to detect and monitor motion and/or pressure of skin-application portion 40 across the skin as described hereinabove with reference to FIG. 1.

Reference is made to FIGS. 5A-C, which are schematic illustrations of alternative configurations of piezoelectric element 60, for use with the apparatus for skin treatment (as described with reference to any of the other figures), in accordance with some applications of the present invention. For some applications, transducer 50 comprises a single piezoelectric element 60 having a plurality of distinct active areas. Such a configuration of piezoelectric element 60 typically allows use of fewer (e.g., one) elements 60 in transducer 50. Typically, having fewer (e.g., one) piezoelectric elements 60 simplifies the manufacturing process of transducer 50 and reduces manufacturing cost. For some applications, transducer 50 comprises more than one piezoelectric element 60.

FIG. 5A shows a piezoelectric element 60 for use in apparatus 20, comprising a plurality of conductive strips 74 which coat or are applied to the piezoelectric element as strips of conductive material with electrode functionality, e.g., strips of silver and/or gold. Piezoelectric element 60 may be configured to provide transmission of ultrasound energy in a phased array mode that typically allows changing the depth of a focal zone.

FIG. 5B shows piezoelectric element 60 having coated, i.e., active, areas 15 and non-coated, i.e., non-active, areas 16. Such a design of piezoelectric element 60 typically allows application of less energy to a desired skin area.

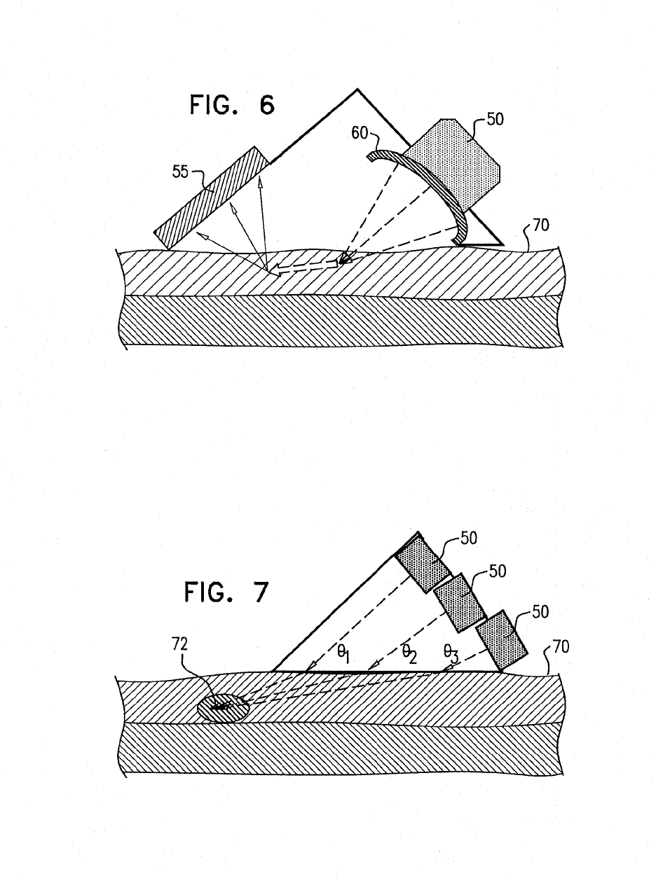

FIG. 5C shows piezoelectric element 60 having active area 15 which may be coated, and non-active area 16 which is typically not coated. For some applications, piezoelectric element 60 comprises a first area 18 which represents an active area for transmitting low intensity ultrasound energy. Typically, a second area is located on an opposite side of piezoelectric element 60, for receiving the low intensity ultrasound energy from first area 18. Analysis of the received signal can be used to estimate the treatment effect or the temperature reached in the skin, e.g., using the techniques described hereinbelow with reference to FIG. 6.

It is to be noted that piezoelectric element 60 is described with reference to FIGS. 2-3 and 5A-C by way of illustration and not limitation, and that any other suitable type of piezoelectric element 60 and/or transducer 50 may be used.

It is noted that apparatus 20 is typically configured to monitor a temperature of the treated skin. This may be achieved by attaching a thermal sensor to the apparatus 20 and/or by transmitting ultrasound energy from an active area of piezoelectric element 60 and receiving the transmitted energy by another active area. Various acoustic parameters are dependent on the temperature of the tissue. For example, the speed of sound (and, correspondingly, time of flight (TOF)) is dependent on the temperature of the tissue, so changes in the speed of sound as indicated by time of flight measurements may be used to estimate a change in skin temperature. Additional acoustic parameter such as signal amplitude and or sub-harmonics of the reflected ultrasound energy can be analyzed to estimate temperature change.

With reference to any of the figures, it is noted that apparatus 20 is typically configured to apply energy to treat an inner layer of the skin, e.g., the dermis, substantially without affecting the outer layer of the skin, the epidermis. For some applications, transducer 50 is configured to transmit focused or non-focused ultrasound energy at a particular angle, such that the ultrasound waves are transmitted to the skin at a desired depth thereby affecting desired skin layers, e.g., the dermis, generally without affecting the outer layers. For example, transducer 50 may be configured to transmit ultrasound energy at a critical angle with respect to the skin, or at an angle that is close to the critical angle or smaller than the critical angle, such that the transmitted waves run substantially parallel to the outer skin surface.

Reference is made to FIG. 6. For some applications, apparatus 20 comprises more than one acoustic element. Transducer 50 is shown comprising an arched piezoelectric element 60 as described hereinabove. Apparatus 20 is shown to comprise an additional acoustic element 55, e.g., a receiver or a reflector. For some applications, a portion of the ultrasound energy waves transmitted by transducer 50 are received and/or reflected by acoustic element 55 to monitor the changes in skin temperature as described hereinabove, e.g., by calculating time of flight from transmission of an ultrasound pulse by piezoelectric element 60 until it is received by acoustic element 50, or by calculating time of flight from transmission of an ultrasound pulse by piezoelectric element 60 until it is reflected by acoustic element 50 and subsequently received by piezoelectric element 60.

For some applications, as shown in FIG. 6, transducer 50 may be configured to transmit ultrasound energy at an angle that is generally smaller than the critical angle, to generally reduce reflection of the transmitted waves, such that the transmitted waves penetrate the skin and run generally parallel to the outer skin surface and slightly into the dermis.

Reference is made to FIG. 7, which is a schematic illustration of yet another configuration of apparatus 20 for skin treatment, in accordance with some applications of the present invention. Apparatus 20 is shown comprising an array of transducers 50, e.g., 3-30 transducers 50. The array of transducers 50 shown in FIG. 7 typically comprises flat transducers, each transmitting at a different angle such that the transmitted ultrasound energy is focused at a desired depth, as shown. For some applications, this desired depth is modified during a procedure using phased array techniques and/or by mechanical aiming of the elements, in order to cause heating at more than one depth or site in the skin.

Reference is made to FIGS. 1-7. It is to be noted that piezoelectric element 60 as described with reference to FIGS. 5A-B can be used in the apparatus of FIGS. 1-4 and 6. Additionally or alternatively, any transducer 50 described with reference to FIGS. 1-7 may be used in apparatus 20 and in combination with any element of apparatus 20 described herein.

With reference to any of the figures, it is to be noted that apparatus 20 is typically relatively simple and inexpensive to manufacture. Thus, apparatus 20 or portions thereof e.g., skin-application portion 40, may be disposable.

With reference to any of the figures, for some applications, a substance containing a heat-sensitive agent is injected through the skin of the subject prior to application of the ultrasound energy thereto. Typically, the ultrasound energy applied to the skin elevates a temperature of the skin or underlying tissue, thereby affecting (e.g., changing a mechanical property of) the heat-sensitive agent in the substance that was injected through the skin. For example, the change in temperature may change the viscosity of the substance, thereby stabilizing the substance below the skin of the subject and/or reducing any discomfort. For some applications, the substance comprises additional agents which may be therapeutic and/or cosmetic agents. For example, the substance may comprise a cosmetic filler including collagen and/or silicone and/or hyaluronic acid. For some applications, the heat-sensitive agent comprises high molecular weight sodium polystyrene sulfonate.

With reference to any of the figures, apparatus and methods described herein may be practiced in combination with techniques for assessing a parameter of tissue described in the following articles: Moran C M et al., entitled “Ultrasonic propagation properties of excised human skin,” Ultrasound Med Biol. 21(9):1177-90 (1995) and Akashi N et al., entitled “Acoustic properties of selected bovine tissue in the frequency range 20-200 MHz,” J Acoust Soc Am. 98(6):3035-9 (1995), which are incorporated herein by reference.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus, comprising:
a skin-application portion, configured to move across skin of a subject;
at least one acoustic element coupled to the skin-application portion and configured to be placed in acoustic contact with the skin, and configured to apply ultrasound energy to the skin;
circuitry configured to generate a current responsive to motion of the skin-application portion; and
a control unit, which is configured to receive the current, to determine, responsive thereto, whether the skin-application portion is moving with respect to the skin, and to drive the acoustic element to apply the ultrasound energy to the skin responsive to determining that the skin-application portion is moving with respect to the skin.

2. The apparatus according to claim 1, further comprising a pressure sensor configured to generate a pressure sensor signal responsive to contact of the skin-application portion with the skin, and wherein the control unit is adapted to receive the pressure sensor signal, to determine, responsive thereto, a degree of contact of the skin-application portion with the skin, and to control application of ultrasound energy from the acoustic element to the skin responsive to determining the contact of the skin-application portion with the skin.

3. The apparatus according to claim 1, further comprising at least one rotating element coupled to the circuitry.

4. (canceled)

5. The apparatus according to claim 1, wherein the rotating element comprises a wheel that is rotatable due to the skin-application portion being moved with respect to the skin, while in contact with the skin, and wherein the wheel is permitted to turn in a first direction, and inhibited from turning in a second direction, opposite to the first direction.

6. The apparatus according to claim 5, wherein the control unit is configured to allow the application of the ultrasound energy when the wheel turns in a first direction, and to inhibit the application of the ultrasound energy when the wheel is turning in a second direction, opposite to the first direction.

7. (canceled)

8. The apparatus according to claim 1, wherein the skin-application portion is configured to move across the skin in a continuous manner.

9. The apparatus according to claim 1, wherein the acoustic element comprises an ultrasound transducer configured to apply the ultrasound energy as high intensity focused ultrasound (HIFU) energy.

10-11. (canceled)

12. The apparatus according to claim 1, wherein the acoustic element comprises a piezoelectric element and wherein the apparatus further comprises a conductive material coupled to a plurality of distinct areas of the same piezoelectric element.

13. The apparatus according to claim 12, wherein a first one of the plurality of distinct areas is configured to receive ultrasound energy transmitted by a second one of the distinct areas, and wherein the control unit is configured to determine an indication of a temperature of the skin, in response to a parameter of the received energy, and to regulate the application of the ultrasound energy in response to the parameter.

14-15. (canceled)

16. The apparatus according to claim 1, wherein the at least one acoustic element comprises at least two acoustic elements, respectively comprising an ultrasound transmitter and an ultrasound receiver.

17. The apparatus according to claim 16, wherein the ultrasound receiver is configured to receive reflected ultrasound energy and the apparatus is configured to monitor an ultrasound parameter of the reflected energy.

18. A method comprising:
moving an acoustic element across skin of a subject, while the acoustic element is in acoustic contact with the skin;
activating the acoustic element to apply ultrasound energy to the skin;
generating a current responsive to motion of the acoustic element across the skin;
determining, responsive to the current, whether the acoustic element is moving with respect to the skin; and
controlling application of ultrasound energy from the acoustic element to the skin responsive to determining that the acoustic element is moving with respect to the skin.

19. The method according to claim 18, further comprising operating a pressure sensor to generate a pressure sensor signal responsive to the contact of the acoustic element with the skin, and wherein determining further comprises determining, in response to the pressure signal, a degree of contact of the acoustic element with the skin, and controlling application of ultrasound energy from the acoustic element to the skin responsive to determining the contact of the acoustic element with the skin.

20-21. (canceled)

22. The method according to claim 18, further comprising monitoring a temperature of the skin in response to activating the acoustic element to apply ultrasound energy to the skin.

23. The method according to claim 18, further comprising:
applying a substance to the skin, the substance including an agent; and
facilitating delivery of the agent into the skin by the application of the ultrasound energy.

24. The method according to claim 18, further comprising:
applying a substance to the skin, the substance including an agent; and
activating the agent by the application of the ultrasound energy.

25-26. (canceled)

27. The method according to claim 24,
wherein the substance includes metal nano-particles, and
wherein activating the agent by the application of the ultrasound energy comprises raising a temperature of the substance by reflecting the ultrasound energy from the metal particles.

28. (canceled)

29. Apparatus, comprising:
a skin-application portion, configured to move across skin of a subject;
at least one energy source coupled to the skin-application portion and configured to be placed in contact with the skin, and configured to apply energy to the skin;
circuitry configured to generate a current responsive to motion of the skin-application portion; and
a control unit, which is configured to receive the current, to determine, responsive thereto, whether the skin-application portion is moving with respect to the skin, and to drive the energy source to apply the energy to the skin responsive to determining that the skin-application portion is moving with respect to the skin.

30. (canceled)

31. A substance for application to skin of a subject, comprising:
an agent; and
microcapsules containing the agent and configured to release the agent into the skin by rupturing in response to ultrasound energy.

32-34. (canceled)

35. A substance for application to skin of a subject comprising:
metal particles configured to raise a temperature of the substance by reflecting ultrasound energy applied to the skin; and
an agent configured to assume an active state thereof in response to the raise in the temperature of the substance.

36-38. (canceled)
